(19) United States
(12) Patent Application Publication
(10) Pub. No.: US 2019/0351221 A1
(43) Pub. Date: Nov. 21, 2019

(54) LEAD PADDLE POSITIONING AND/OR DEPLOYMENT SYSTEM, A LEAD PADDLE AND A STYLET

(71) Applicant: GTX medical B.V., Eindhoven (NL)

(72) Inventors: Damian GANTY, Eindhoven (NL); Vincent DELATTRE, Eindhoven (NL)

(21) Appl. No.: 16/415,867

(22) Filed: May 17, 2019

(30) Foreign Application Priority Data
May 18, 2018 (EP) 18173218.1

(51) Int. Cl. A61N 1/05

(52) U.S. Cl. CPC A61N 1/0553; A61B 17/3468; A61N 1/0558

(57) ABSTRACT

The present invention relates to a lead paddle positioning and/or deployment system comprising at least a stylet and a lead paddle, wherein the lead paddle comprises at least one guiding channel and wherein the stylet comprises at least one guiding wire at its distal end, which is configured and arranged to be engaged and/or to be at least partially received in the guiding channel. The present invention relates further to a stylet for a lead paddle system comprising the stylet features. Furthermore, the present invention relates to a lead paddle for a lead paddle system comprising the lead paddle features.

### CROSS-REFERENCE TO RELATED APPLICATION

The present application claims priority to European Application No. 18173218.1 entitled “A LEAD PADDLE POSITIONING AND/OR DEPLOYMENT SYSTEM, A LEAD PADDLE AND A STYLET,” filed May 18, 2018. The entire contents of the above-identified application are hereby incorporated by reference for all purposes.

### TECHNICAL FIELD

The present invention relates to a lead paddle positioning and/or deployment system comprising at least a stylet and a lead paddle, a stylet and a lead paddle.

### BACKGROUND AND SUMMARY

At the moment it seems that the existing technique and surgical procedures do not allow placing easily a lead paddle at the right and intended place into the epidural space, especially for long and wide arrays/paddles.

This technique might be helpful especially for “difficult cases” such as patients with scar tissues, fibrosis, calcification due to previous surgeries or lead replacement. The stylet would help to safely steer and push the paddle into the canal at the intended place.

Some single lumen stylet may already exist for the need insertion but there is also a trade-off between pushabilities/steerabilities and safety due to stylet material stiffness diameter limits.

Lead paddles are for example known from U.S. Pat. No. 8,108,051 B2, US 2013/0096662 A1, US 2012/0006793 A1 and EP 3 013 411 A1.

A stylet system is for example known from U.S. Pat. No. 6,309,401 B1 or from US 2003/0135253 A1, which relates to a neurostimulation lead stylet handle.

It is therefore an object of the present invention to provide a lead paddle positioning and/or deployment system, a stylet for a lead paddle system and a lead paddle, in particular in that the axial stiffness and radial flexibility of a lead paddle is increased and a safe and accurate positioning of the lead paddle can be insured.

This object is solved according to the present invention by a lead paddle positioning and/or deployment system with the features of claim 1.

Accordingly, a lead paddle positioning and/or deployment system is provided, at least a stylet and a lead paddle, wherein the lead paddle comprises at least one guiding channel and wherein the stylet comprises at least one guiding wire at its distal end, which is configured and arranged to be engaged and/or to be at least partially received in the guiding channel.

The invention is based on the basic idea that by providing a guiding channel the form and stability of the lead paddle can already be influenced in terms of axial stiffness and radial flexibility. For easier deployment a stylet is provided, which allows maneuvering and handling of the whole system via a minimally invasive incision in connection with the implantation side.

In particular, the guiding channel may be U-shaped.

The guiding channel with its U-shaped form may have a round part and a closed part at its distal end and at its proximal two ends.

The two ends may allow introducing a guiding wire. The guiding wire may be the guiding wire of the stylet.

Furthermore, it is possible that the lead paddle comprises two guiding channels. By providing two guiding channels, more options for introducing the guiding wires of a stylet may be provided.

The guiding channels may extend over more then a half of the length of the length of the lead paddle, especially it is possible that the guiding channel extends over more then half or more then 80% of the length of the lead paddle.

In particular, it is possible that the guiding channel(s) is/are arranged along the outer edge region of the lead paddle. In particular, if the outer form of the lead paddle and the outer edge of the lead paddle already comprises an elongated form with a round tip, then with a U-shape the guiding channel is following the form of the outer edge of the lead paddle.

Furthermore, the guiding channels may be connected by a connection section, such that the overall form of the guiding channels with the connection section is forming a connected guiding channel structure, which is U-shaped. By this, the form of the guiding channel structure enhances the overall stability of the lead paddle and provides additional radial flexibility in connection with axial stiffness.

The guiding wire may be flexible and/or bendable. By this, the guiding wire can be inserted more easily into a guiding channel or a guiding channel structure and also follow the guiding channel or the guiding channel structure.

For example, the guiding wire can be inserted by sliding into the guiding channel structure, such that the stylet wire can be single and inserted/removed from one extremity of the guiding channels and exit from the other extremity of the guiding channels, the overall shape of the guiding wire is then forming a U-shape in the guiding channel structure.

The stylet may comprise two guiding wires. By two guiding wires better steering of the lead paddle, when engaged with the stylet, is possible. Furthermore, it is possible that the stylet may have a fork-like form. By this, the engagement with the guiding channel and the guiding wire is easier possible. The guiding wires or the guiding wire may be dis-engageable with the stylet.

Furthermore, the stylet may comprise a U-shaped guiding-wire receiving portion. With such a U-shaped guiding wire receiving portion stability on the one hand and a better introduction of forces over moments and bending forces and flexibility can be provided.

The stylet may also comprise a handle, which may be connected to the U-shaped guiding-wire receiving portion. By means of the handle a better handling of the overall stylet may be provided. The handle may be made out of a stiff material such as metal of a re-enforced or stiff polymer/plastic.

Furthermore, it is possible that the guiding-wire receiving portion is curved. By this it is possible to allow easier deployment of the lead paddle via the incision during implantation.

The lead paddle positioning and/or deployment system may also be characterized in that the curvature of the guiding-wire receiving portion is less than the curvature of the curved connecting portion. This results in a form that the engaged stylet with the lead defines an angle of more than 90 degree, which is beneficial for implantation.

In particular, it is possible that the stylet has a proximal end, which defines with the distal end of the guiding wire an angle in the range of approx. 80° to 100°.

Moreover, it is possible that in the engaged state the lead paddle and the stylet have a curvature radius of the lead induced by the engagement with the guiding wires is approx. 90 to 110 mm, especially approx. 100 mm, and/or that the angle between the lead paddle and the stylet, especially the U-shaped guiding-wire receiving portion, is chosen in the range of approx. 5° to 15°, especially at approx. 10°.

The guiding-wire receiving portion may be configured and formed such that in combination with a predetermined length of the guiding wire a portion of the guiding-wire receiving portion forms a mechanical stop, which reaches one edge of the lead paddle, when engaging the lead paddle and stylet. By this it can be prevented that the mechanical stop comes from the guiding wire, when inserting the guiding wire into the guiding channel of the lead paddle. This will help to avoid for example punctures of the lead paddle or the like.

Furthermore, the lead paddle positioning and/or deployment system comprises at least one lead body and a lead body connection portion, wherein the lead body connection portion is arranged on a flat side of the lead paddle with an offset to the edge of the proximal end of the lead paddle, especially centric with regard to the axial axis of the lead paddle, and wherein the lead body is connected to the lead paddle in the lead body connection portion. There may be one or more lead bodies. Possible embodiments may comprise for example two lead bodies. The lead bodies can be arranged centric with regard to the axial axis of the lead paddle and connected on the opposite side of the contact side of the lead paddle, i.e. the upper side of the lead paddle. The lead body connection portion may be arranged with an offset to the edge of the proximal end of the lead paddle, such that the lead paddle may be moved back and forth and also to the left and right and vice versa even after deployment in the spinal canal.

The present invention also relates to a stylet for a lead paddle system as disclosed above.

Furthermore, the present invention also relates to a lead paddle for a lead paddle positioning and/or deployment system as disclosed above.

The lead paddle may especially have a U-shaped guiding channel. The guiding channel may be arranged along the outer edge region of the lead paddle.

The stylet material and diameter may be stiff enough to allow pushing and steering in the canal, i.e. the guiding wire.

The stylet material and diameter may be soft enough to be easily removable. For this, a coating such as Teflon might be very helpful and be used.

The U-shape main feature gives the “safety” compared to the single lumen stylet ball tip extremity.

The feature to have two stylet lumens on paddle sides instead of having only one at the center gives more steering and driving possibilities.

Furthermore, the above construction allows easy unclipping the handle from the stylet removal.

Also a ball tip at the stylet extremity is foreseen for a safe removal.

Furthermore, the stylet may be pre-bended with an adequate angle for insertion to the canal.

This allows more freedom of movement and better handling in the angle may be adjusted by the user insertion more easily.

### BRIEF DESCRIPTION OF THE FIGURES

Further details and advantages of the present invention shall now be disclosed in connection with the drawings.

It is shown in

FIG. 1 a first embodiment of a lead paddle;

FIG. 2 a lead paddle positioning and deployment system with the lead paddle of FIG. 1;

FIG. 3 a schematical view of the deployment of the lead paddle by means of the lead paddle positioning and deployment system;

FIG. 4 a further schematical view of the deployment of the lead paddle by means of the lead paddle positioning and deployment system;

FIG. 5 a further embodiment of a lead paddle positioning and deployment system according to the present invention;

FIG. 6 a further view of the lead paddle positioning and/or deployment system according to FIG. 5;

FIG. 7 a further view of the lead paddle positioning and/or deployment system according to FIG. 5;

FIG. 8 a further view of the lead paddle positioning and/or deployment system according to FIG. 5; and

FIG. 9 a second embodiment of a lead paddle according to the present invention.

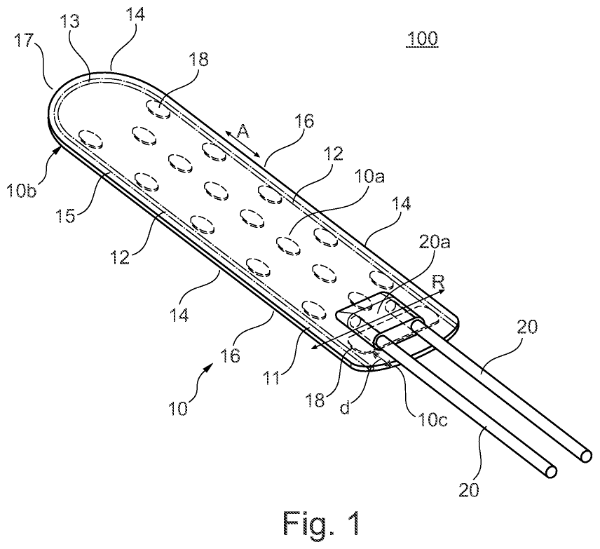

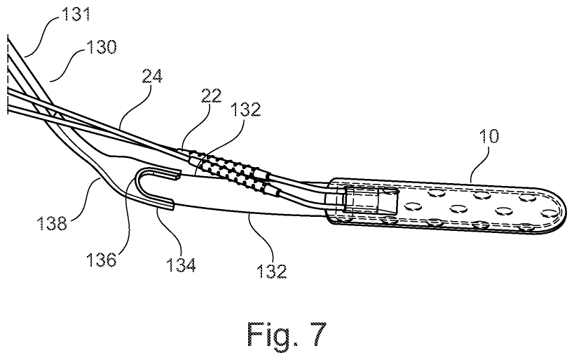

It will be appreciated that FIGS. 1-9 show example configurations with relative positioning of the various components. Further, the components are shown to scale. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example.

### DETAILED DESCRIPTION

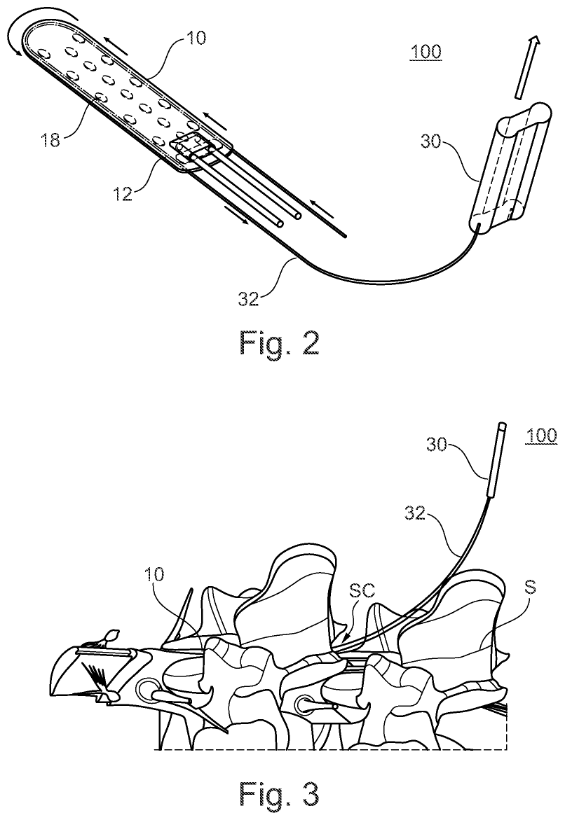

FIG. 1 shows in a perspective view a possible embodiment of a lead paddle 10 according to the present invention of a lead paddle positioning and deployment system 100 (cf. FIG. 2) according to the present invention.

The lead paddle 10 comprises a lead paddle body 11 and comprises two guiding channels 12.

The two guiding channels 12 are embedded in the lead paddle body 11.

The lead paddle body 11 may be made of a medical grade material, such as a medical grade polymer. In particular, a medical grade silicone or the like may be used.

The guiding channels 12 extend over more then half of the length of the length of the lead paddle 10.

Especially, the guiding channels 12 extend over more then a half of 80% of the length of the lead paddle 10.

In particular, the guiding channels 12 are arranged along the outer edge region 14 of the lead paddle 10.

In particular, the guiding channels 12 are arranged parallel to the longitudinal edge 16 of the lead paddle 10.

The lead paddle 10 comprises a plurality of electrodes 18.

The electrodes 18 are embedded in the body of the lead paddle 10.

The shape of the electrodes 18 is rectangular.

The shape of all electrodes is more or less identical. Here the electrodes 18 are identical in their form.

Generally speaking, the form of one or more electrodes can be designed differently. In particular, they can be oval, round, square, diamond shape, trapezoidal or the like.

The arrangement of the electrodes 18 is also such that some electrodes are offset to each other.

The electrodes 18 of the lead paddle 10 are connected to lead bodies 20.

The lead bodies 20 are connected to a connection portion 20a on the upper side 10a of the lead paddle 10.

This connection portion 20a is on the opposite side of the contact side 10b of the lead paddle 10, that is configured and arranged to get in contact with the tissue to be stimulated, i.e. here the spinal cord of the patient.

Furthermore, the connection portion 20a is arranged centric with regard to the axial axis of the lead paddle 10. Moreover, the connection position 20a is positioned with an offset d to the edge of the proximal end 10c of the lead paddle 10.

As the connection portion 20a of the lead bodies 20 is not directly arranged at the outer edge of the lead paddle 10, the deployment and positioning of the lead paddle 10 is enhanced.

In particular, the offset d helps that by means of the lead bodies 20 the lead paddle 10 may be moved back and forth and also to the left and right and vice versa even after deployment in the spinal canal. As the lead bodies are arranged on the upper side with an offset to the edge, the proximal edge of the lead paddle is free and especially a movement in the proximal direction is not obstructed by the lead bodies.

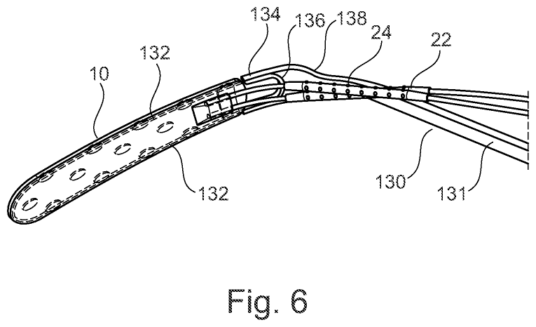

As can be seen in FIG. 6 to FIG. 8, the lead bodies 20 can be provided with anchoring sleeves 22.

The anchoring sleeves 22 are attached to the outer side of the lead body 20.

Furthermore, the anchoring sleeves 22 are provided with pins 24, which extend radially from the outer side of the anchoring sleeve 22.

The anchoring sleeves 22 are not shown in FIG. 1.

Moreover, the two guiding channels 12 are extended to the tip end 17 of the lead paddle body 11 with a connection section 13, such that the overall form of the guiding channels 12 with the connection section is forming a connected guiding channel structure 15, which is U-shaped.

The guiding channels 12 alone, but also the guiding channel structure 15 is configured and arranged such that guiding wires of a stylet may be received in the guiding channels 12 and thus stylet and lead paddle 10 may be engaged with each other for deployment and positioning.

The lead paddle body 11 is flexible by means of the chosen material, but also by its structure. However, the flexibility of the lead paddle body 11 is different in axial direction A compared to the radial direction R.

Stiffness in the axial direction A is provided by the electrodes 18 in connection with the guiding channels 12 and the U-shaped guiding channel structure 15.

Surprisingly, it was found in experimental setups and lead paddle deployments that at least one of the guiding channels 12 already and significantly enhances the axial stiffness, but does not affect the radial flexibility.

So, the paddle body 11 and thus the lead paddle 10 comprises axial stiffness in the direction A and radial flexibility in the direction R.

So, it is generally possible that even without a stylet and by means of the lead bodies 20 the lead paddle 10 can be inserted into the spinal channel. For such an insertion axial stiffness is necessary to avoid bending in axial direction, whereas (slight) bending in the radial direction is wanted to adapt to the anatomical structures at the implantation site in the spinal channel.

FIG. 2 shows the embodiment of FIG. 1 of the lead paddle 10 in connection with the possible embodiment of a stylet 30 and a guiding wire 32.

The stylet 30 comprises at least one guiding wire 32 at its distal end, which is configured and arranged to be engaged and received by the guiding channel 12.

The guiding wire 32 is flexible and bendable.

Reference is also made to FIG. 5-9, where for example two guiding wires 32′ may be used (see also the embodiment of FIGS. 5 to 8).

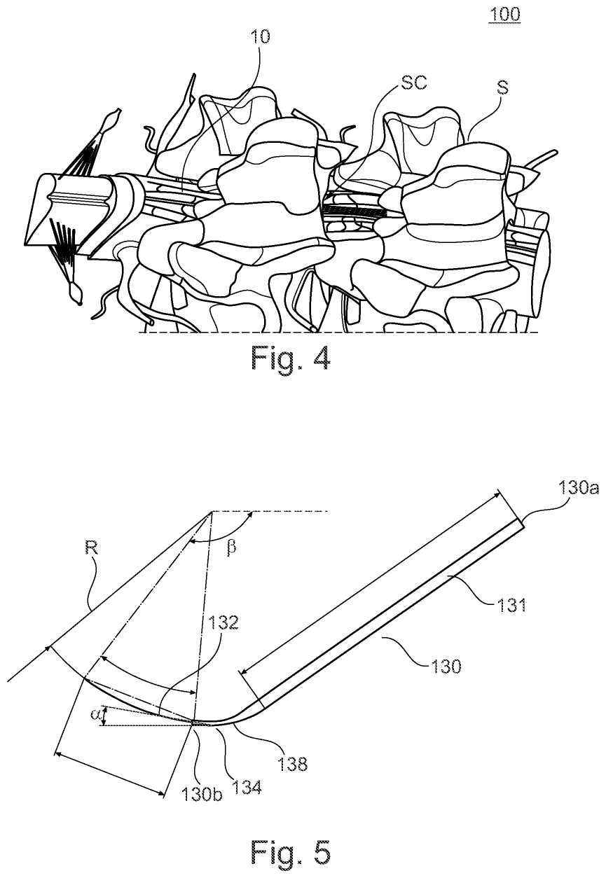

FIG. 3 and FIG. 4 show schematical views of the deployment of the lead paddle 10 by means of the lead paddle positioning and deployment system 100 into the spinal channel SC of the spinal column S.

The stylet 30 is during the deployment and positioning of the lead paddle 10 engaged with the lead paddle 10 by means of the lead wire 32.

The guiding wire 32 is in the situation shown in FIG. 3 positioned and engaged in and with the guiding channel structure 15 of the lead paddle 10.

For engagement, the flexibility and bendability of the guiding wire 32 is used. By this, the guiding wire can be inserted more easily into the guiding channel structure 15 and also follow the guiding channel structure 15.

Here, the guiding wire can be inserted by sliding into the guiding channel structure, such that the stylet wire can be single and inserted/removed from one extremity of the guiding channels and exit from the other extremity of the guiding channels, the overall shape of the guiding wire is then forming a U-shape in the guiding channel structure.

So, the stylet may be moved towards the spinal column S and thereby the lead paddle 10 is pushed and moved forward in the spinal channel SC to the correct implantation position.

The positioning is shown in FIG. 3.

After reaching the correct implantation position, the guiding wire 32 is disengaged with the guiding channel structure 15 of the lead paddle 10. Then the stylet 30 and the guiding wire 32 are removed and the lead paddle 10 is left in the spinal channel SC.

FIG. 4 shows the correct positioned lead paddle 10 in the spinal channel SC.

FIG. 5 shows a further embodiment of a stylet 130 of/for the lead paddle positioning and deployment system 100.

Each reference number of the stylet 30 according to FIG. 2 and FIG. 3 is in the embodiment shown in FIG. 5 to FIG. 8 increased by the value 100 for similar or identical features of the stylet 30 according to FIG. 2 and FIG. 3.

The stylet 130 comprises each and every structural and functional feature as the stylet 30 according to FIG. 2 and FIG. 3. It can be used similar and/or identical like the stylet 30 as described above in connection with FIG. 2 and FIG. 3.

However, the stylet 130 as shown FIG. 5 to FIG. 8 comprises the following differences:

The stylet 130 has a handle 131.

The handle 131 may be made of a stiff material, like a stiff polymer or even a metal.

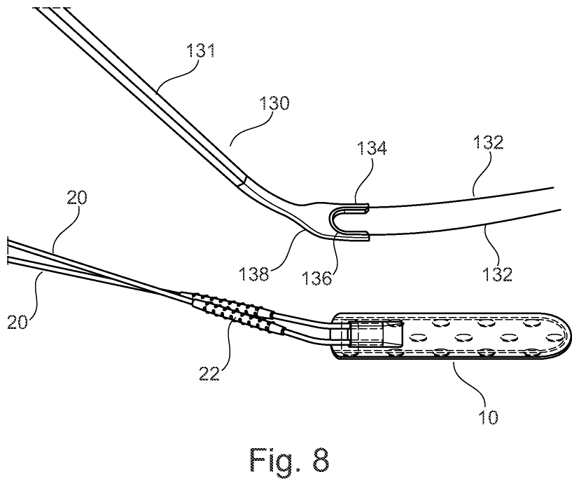

In FIG. 5 to FIG. 8, it becomes clear that the stylet 130, which is suitable for the embodiment of the lead paddle 10 or 10′, has a fork like form (cf. e.g. FIG. 8).

Furthermore, the stylet 130 comprises a U-shaped guiding wire receiving portion 134.

The handle 131 is connected to the U-shaped guiding-wire receiving portion 134.

The guiding wire 132 itself may also be U-shaped and being received in a U-shaped receiving recess 136 within the U-shaped guiding wire receiving portion 134.

As the guiding wire 132 is flexible and bendable, the U-shaped form of the guiding wire 132 is achieved by inserting the guiding wire 132 into the receiving recess 136.

As the guiding wire 132 has two free ends, the stylet 130 comprises by this arrangement two guiding wires 132 provided by the free ends of the guiding wire 132 extending out of the U-shaped receiving recess 136.

The U-shaped guiding-wire receiving portion 134 and the handle 131 are connected to each other with a curved connecting portion 138.

The guiding wire receiving portion 134 is curved.

Furthermore, the curvature of the guiding wire receiving portion 134 is less than the curvature of the curved connecting portion 138.

FIG. 5 also shows details on angles and radius of the design of the stylet 130.

The stylet 130 has a proximal end 130a and a distal end 130b.

The proximal end 130a defines with the distal end 130b, but also with the distal end of the guiding wire 132 an angle β in the range of approx. 80° to 100°.

The guiding wire receiving portion 134 is configured and formed such that in combination with a predetermined length of the guiding wire 132 a portion of the guiding-wire receiving portion 134 forms a mechanical stop, which reaches one edge of the lead paddle 10, when engaging the lead paddle 10 and stylet 130.

In the engaged state of the lead paddle 10 and the stylet 130, the curvature radius R of the lead paddle 10 induced by the engagement with the guiding wires 132 is approx. 90 to 110 mm, here in the shown embodiment of FIG. 5 it is 100 mm.

The angle α between the lead paddle 10 and the stylet 130, here with the U-shaped guiding-wire receiving portion 134, is chosen in the range of approx. 5° to 15°, here at 10°.

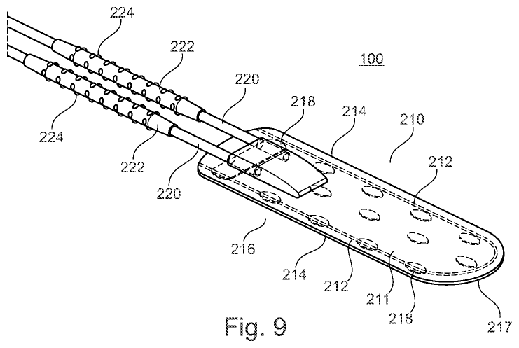

FIG. 9 shows a further embodiment of a lead paddle 210 for the lead paddle positioning and deployment system 100.

Each reference number of the lead paddle 10 according to FIG. 1 is in the embodiment shown in FIG. 9 increased by the value 200 for similar of identical features of the lead paddle 10 according to FIG. 1.

The lead paddle 210 comprises each and every structural and functional feature as the lead paddle 10 according to FIG. 1.

However, the lead paddle 210 comprises the following differences:

The lead paddle 210 comprises two separate guiding channels 212.

The guiding channels 212 are not connected to each other.

The guiding channels 212 are also parallel to each other and completely straight.

On each lead body 220 have one anchoring sleeve 222 with the pins 224 is arranged.

### REFERENCES

10 lead paddle
10a upper side
10b contact side
10c proximal end
11 lead paddle body
12 guiding channel
13 connecting portion
14 outer edge region
15 guiding channel structure
16 longitudinal edge
17 tip end
18 electrodes
20 lead body
20a connection portion
22 anchoring sleeves
24 pins
30 stylet
32 guiding wire
100 lead paddle positioning and deployment system
130 stylet
130a proximal end of the stylet
130b distal end of the stylet
131 handle
132 guiding wire
134 U-shaped guiding wire receiving portion
136 U-shaped receiving recess
138 curved connecting portion
210 lead paddle
211 lead paddle body
212 guiding channel
214 outer edge region
216 longitudinal edge
217 tip end
218 electrodes
220 lead body
222 anchoring sleeves
224 pins
d offset
R radius
S spinal column
SC spinal channel
α angle
β angle

1. A lead paddle, comprising:
a paddle body;
at least one guiding channel for receiving a guiding wire, the at least one guiding channel arranged along an outer edge region of the paddle body and extending over more than half of a length of the lead paddle.

2. The lead paddle of claim 1, wherein the at least one guiding channel follows a form of the outer edge region.

3. The lead paddle of claim 1, wherein the at least one guiding channel includes two guiding channels, one of the two guiding channels arranged along a first side of the outer edge region of the paddle body, a remaining of the two guiding channels arranged along a second, opposite side of the outer edge region.

4. The lead paddle of claim 3, wherein the outer edge region has a tip, and wherein the tip is a round tip, and wherein the paddle is shaped to fit in a spinal channel.

5. The lead paddle of claim 3, further comprising a connection section connecting the two guiding channels.

6. The lead paddle of claim 5, wherein an overall form of the two guiding channels connected via the connection section is U-shaped.

7. The lead paddle of claim 1, wherein the at least one guiding channel extends over more than 80% of the length of the lead paddle.

8. The lead paddle of claim 3, further comprising a plurality of identically shaped electrodes embedded in the paddle body.

9. The lead paddle of claim 8, further comprising a U-shaped guiding-wire receiving portion arranged on an upper side of the paddle with an offset from a proximal end of the paddle, the proximal end opposite the tip of the paddle, a lower side of the paddle configured to be in contact with a tissue to be stimulated by the plurality of identically shaped electrodes of the paddle.

10. The lead paddle of claim 9, wherein a flexibility of the paddle in an axial direction, from the proximal end to the tip of the paddle, is different from the flexibility of the paddle in a radial direction, from the first side to the second side of the outer edge region.

11. The lead paddle of claim 10, wherein the flexibility of the paddle is higher in the radial direction than in the axial direction.

12. A lead paddle, comprising:
a paddle body defined by a proximal end, a tip end, and a pair of longitudinal edges;
two guiding channels for receiving a guiding wire, a first of the two guiding channels arranged along a first of the pair of longitudinal edges of the paddle body, a second of the two guiding channels arranged along a second of the pair of longitudinal edges of the paddle body;
a connection portion connecting the two guiding channels to each other at the tip end; and
a plurality of electrodes embedded in the paddle body and arranged with an offset to one another.

13. The lead paddle of claim 12, wherein the tip end is round and wherein the two guiding channels with the connection portion form a U-shaped guiding channel structure extending over more than half of a length of the lead paddle.

14. The lead paddle of claim 12, further comprising:
a lead body connection portion arranged on an upper side of the paddle with an offset to the proximal end of the paddle; and
one or more lead bodies coupled to the plurality of electrodes via the lead body connection portion, the one or more lead bodies configured to receive a guiding wire for positioning the lead paddle.

15. The lead paddle of claim 14, wherein the lead body connection portion is U-shaped and curved.

16. The lead paddle of claim 14, wherein the one or more lead bodies are arranged centric to an axial axis of the lead paddle, the axial axis running from the tip end to the proximal end of the paddle.

17. The lead paddle of claim 16, wherein the paddle has a higher stiffness along the axial axis than along a radial axis, the radial axis connecting the pair of longitudinal edges to each other.

18. A method for a lead paddle, comprising:
receiving a guiding wire in a guiding channel arranged along an outer edge region of a body of the lead paddle, the guiding channel extending over more than half a length of the lead paddle;
steering the lead paddle into a spinal channel at an implantation position via the received guiding wire; and
energizing a plurality of identically shaped electrodes embedded in the body of the paddle to stimulate tissue in the spinal channel.

19. The method of claim 18, further comprising, following deployment of the paddle in the spinal channel, moving the paddle back, forth, left, and/or right via lead bodies coupled to the paddle at a lead body connection portion arranged on an upper side of the paddle with an offset to a proximal end of the paddle.

20. The method of claim 18, wherein receiving the guiding wire in the guiding channel includes receiving a first guiding wire in a first guiding channel arranged on a first longitudinal edge of the paddle body while receiving a second guiding wire in a second guiding channel arranged on a second, opposite longitudinal edge of the paddle body, the first guiding channel coupled to the second guiding channel via a connection section arranged on a distal end of the paddle.
